(19) United States
(12) Patent Application Publication
(10) Pub. No.: US 2026/0217765 A1
(43) Pub. Date: Jul. 30, 2026

(54) NOVEL ANTIMICROBIAL PEPTIDE DESIGNED FROM DUCK-DERIVED ANTIMICROBIAL PEPTIDE ANDUSE THEREOF

(71) Applicant: INDUSTRY-ACADEMIC COOPERATION FOUNDATION, CHOSUN UNIVERSITY, Dong-gu, Gwangju (KR)

(72) Inventors: Yoon Kyung PARK, Jangseong-gun, Jeollanam-do (KR); Min Kyung KIM, Hwasun-gun, Jeollanam-do (KR); Sa Hong LEE, Songpa-gu, Seoul (KR)

(73) Assignee: INDUSTRY-ACADEMIC COOPERATION FOUNDATION, CHOSUN UNIVERSITY, Dong-gu, Gwangju (KR)

(21) Appl. No.: 19/145,716

(22) PCT Filed: Jan. 11, 2024
(86) PCT No.: PCT/KR2024/000514

§ 371 Date: Jul. 3, 2025

(30) Foreign Application Priority Data
Jan. 18, 2023 (KR) 10-2023-0007581

(51) Int. Cl. C07K 7/08; A23B 2/762; A23K 20/147; A61K 8/64; A61K 38/00; A61Q 17/00

(52) U.S. Cl. CPC C07K 7/08; A23B 2/762; A23K 20/147; A61K 8/64; A61Q 17/005; A61K 38/00; A61K 2800/524

(57) ABSTRACT

The present disclosure relates to a new peptide and, more specifically, to a new peptide prepared by means of substitution or/and deletion of some amino acid sequences of a duck-derived antimicrobial peptide, and a use thereof. The novel peptide according to the present disclosure has excellent antimicrobial activity against gram-negative bacteria such as Escherichia coli, Pseudomonas aeruginosa, and Acinetobater baumannii; gram-positive bacteria such as Staphylococcus aureus, Listeria monocytogenes, and Bacillus cereus; and antibiotic-resistant bacteria, and therefore, as a material having antimicrobial activity, the novel peptide can be useful as a material for coating, packaging, preservatives (cosmetic preservatives, food preservatives, pharmaceutical preservatives), and additives (food additives, cosmetic additives, feed additives), as well as for antibiotics, pharmaceuticals, cosmetics, food, feed, and biopesticides. In particular, the novel peptide of the present disclosure has minimal cytotoxicity and has the advantage of being safe for the human body even for long-term use.

### TECHNICAL FIELD

The present disclosure relates to novel peptides and, more particularly, to novel peptides produced by substituting and/or deleting part of the amino acid sequence of duck-derived antimicrobial peptides, and uses thereof.

### BACKGROUND ART

Antibiotics with antibacterial or antifungal effects are substances that exhibit selective toxicity. That is, antibiotics kill microorganisms while exhibiting low toxicity to the human or animal body and are not inactivated by enzymes in the body. These substances mainly exert their effects by inhibiting mechanisms such as DNA replication, transcription and translation of genetic information, energy transport during transcription, and biosynthesis of cell walls, thereby suppressing the growth of microorganisms.

However, the widespread and indiscriminate use of antibiotics has led to an increase in antibiotic-resistant strains. According to AMR (antimicrobial resistance) hospital antibiotic report of the Centers for Disease Control and Prevention (CDC), approximately 100, 000 people were infected with Methicillin-resistant Staphylococcus aureus (MRSA) in 2006 alone due to the misuse and overuse of antibiotics, and about 20,000 of them died. In 2010, a carbapenem-resistant Enterobacteriaceae (CRE) strain harboring the NDM-1 (New Delhi metallo-beta-lactamase) gene, commonly referred to as a superbacteria, was also discovered in Korea, highlighting the growing threat posed by resistant bacteria. Currently, most research efforts worldwide on infectious diseases focus on the development of antimicrobial substances that selectively inhibit or control pathogenic metabolism or biosynthetic pathways, as well as vaccine development through the cultivation of attenuated pathogens. However, existing antimicrobial agents often inhibit enzymes that also regulate host metabolic and biosynthetic pathways, thereby causing side effects and toxicity, and their use is limited due to resistance issues. Moreover, vaccine development alone cannot provide a rapid response to emerging pathogens. Therefore, there is an urgent need to develop a new paradigm of therapeutics capable of effectively controlling infectious diseases at an early stage, and recently, domestic and international researchers have been focusing on antimicrobial peptides (AMPs) as a potential solution to these problems.

Antimicrobial peptides (AMPs) are components of the immediate and nonspecific defense mechanisms found in virtually all species during infection. These peptides typically carry a net positive charge (+2 to +9) and contain more than 30% hydrophobic amino acid residues. Due to these characteristics, when antimicrobial peptides come into contact with negatively charged bacterial membranes, they can form amphipathic α-helices or β-sheets that enable them to insert into the membrane. Once an antimicrobial peptide enters the membrane of a target bacterium, the positively charged peptide binds to the negatively charged lipid membrane of bacteria, viruses, or fungi, causing membrane destabilization and loss of membrane potential, ultimately leading to microbial death. Naturally occurring antimicrobial peptides have emerged as promising candidates for new antibiotics, as they exhibit antimicrobial activity through mechanisms different from those of conventional synthetic antibiotics, and are therefore expected to overcome the problem of antibiotic resistance.

According to studies conducted to date, over 2,000 antimicrobial peptides have been isolated from animals, plants, and other sources. Representative antimicrobial peptides include cecropin, magainin, bombinin, defensin, tachyplesin, and buforin. These antimicrobial peptides commonly consist of 17 to 24 amino acids and exhibit antimicrobial activity not only against Gram-negative and Gram-positive bacteria, but also against prokaryotes and some have also been reported to be effective fungi; against cancer cells and viruses.

Leading to the present disclosure, intensive and thorough research conducted by the present inventors with the aim of developing novel synthetic peptides with enhanced antimicrobial activity based on previously reported antimicrobial peptides succeeded in synthesize seven dCATH analogs (SEQ ID NOS: 2 to 8) using an amphipathic duck-derived antimicrobial peptide (hereinafter briefly referred to as “dCATH”) as a template and resulted in the finding that the synthesized peptide analogs exhibit enhanced antimicrobial activity and significantly reduced cytotoxicity (negligible cytotoxicity) compared to the parent peptide dCATH.

### DISCLOSURE OF INVENTION

### Technical Problem

Accordingly, an aspect of the present disclosure is to provide an antimicrobial peptide that is safe for the human body and exhibits excellent antimicrobial activity.

Another aspect of the present disclosure is to provide an antibiotic that is safe for the human body and exhibits excellent antimicrobial activity, using the antimicrobial peptide.

Another aspect of the present disclosure is to provide a cosmetic composition that is safe for the human body and exhibits excellent antimicrobial activity, using the antimicrobial peptide.

Another aspect of the present disclosure is to provide a food additive that is safe for the human body and exhibits excellent antimicrobial activity, using the antimicrobial peptide.

Another aspect of the present disclosure is to provide a feed additive that is safe for the human body and exhibits excellent antimicrobial activity, using the antimicrobial peptide.

Another aspect of the present disclosure is to provide a cosmetic preservative or pharmaceutical preservative that is safe for the human body and exhibits excellent antimicrobial activity, using the antimicrobial peptide.

Another aspect of the present disclosure is to provide a biopesticide that is safe for the human body and exhibits excellent antimicrobial activity, using the antimicrobial peptide.

Another aspect of the present disclosure is to provide a quasi-drug composition that is safe for the human body and exhibits excellent antimicrobial activity, using the antimicrobial peptide.

Another aspect of the present disclosure is to provide a method for antimicrobial treatment in subjects other than humans, using the antimicrobial peptide.

### Solution to Problem

In order to achieve the goals, the present disclosure provides an antimicrobial peptide in which: i) the 3^{rd} or 4^{th} amino acid in the amino acid sequence of SEQ ID NO: 1 is substituted with alanine (Ala, A); ii) both the 3^{rd} and 4^{th} amino acids are substituted with alanine (Ala, A); iii) both the 3^{rd} and 4^{th} amino acids are substituted with alanine (Ala, A), and one or more of the 8^{th}, 9^{th}, or 13^{th} amino acids are substituted with lysine (Lys, K); or iv) both the 3^{rd} and 4^{th} amino acids are substituted with alanine (Ala, A), the 8^{th}, 9^{th}, and 13^{th} amino acids are substituted with lysine (Lys, K), and the 19^{th} and 20^{th} amino acids are deleted.

In an embodiment of the present disclosure, the antimicrobial peptide may have the amino acid sequence of any one of SEQ ID NOS: 2 to 8.

In an embodiment of the present disclosure, the antimicrobial peptide may exhibit antimicrobial activity against Gram-negative bacteria, Gram-positive bacteria, or antibiotic-resistant bacteria.

In an embodiment of the present disclosure, the Gram-negative bacteria may include at least one selected from Escherichia coli, Pseudomonas aeruginosa, and Acinetobacter baumannii.

In an embodiment of the present disclosure, the Gram-positive bacteria may include at least one selected from Staphylococcus aureus, Listeria monocytogenes, and Bacillus cereus.

In an embodiment of the present disclosure, the antibiotic-resistant bacteria may be Acinetobacter baumannii having antibiotic resistance.

In an embodiment of the present disclosure, the antimicrobial peptide may have a C-terminus amidated.

The present disclosure also provides an antibiotic including the antimicrobial peptide as an active ingredient.

The present disclosure also provides a cosmetic composition including the antimicrobial peptide as an active ingredient for antimicrobial use.

The present disclosure also provides a food additive including the antimicrobial peptide as an active ingredient for antimicrobial use.

The present disclosure also provides a feed additive including the antimicrobial peptide as an active ingredient for antimicrobial use.

The present disclosure also provides a cosmetic preservative or pharmaceutical preservative including the antimicrobial peptide as an active ingredient.

The present disclosure also provides a biopesticide including the antimicrobial peptide as an active ingredient for antimicrobial use.

The present disclosure also provides a quasi-drug composition for including the antimicrobial peptide as an active ingredient antimicrobial use.

The present disclosure also provides a method for antimicrobial treatment in a subject other than a human, including administering the antimicrobial peptide to the subject.

### Advantageous Effects of Invention

The novel peptides according to the present disclosure exhibit excellent antimicrobial activity against Gram-negative bacteria such as Escherichia coli, Pseudomonas aeruginosa, and Acinetobacter baumannii; Gram-positive bacteria such as Staphylococcus aureus, Listeria monocytogenes, and Bacillus cereus; as well as antibiotic-resistant bacteria. Therefore, the peptides can be effectively used as antimicrobial materials in antibiotics, pharmaceuticals, cosmetics, food, feed, and biopesticides, as well as in other applications such as coatings, packaging materials, preservatives (e.g., cosmetic, food, and pharmaceutical preservatives), and additives (e.g., food additives, cosmetic additives, and feed additives). In particular, the novel peptides of the present disclosure exhibit negligible cytotoxicity, offering the advantage of being safe for the human body even with long-term use.

### BRIEF DESCRIPTION OF DRAWINGS

FIG. 1 shows the results of secondary structure formation analysis of dCATH (control), an antimicrobial peptide, and novel dCATH analog peptides (experimental groups) dCATH-A1, dCATH-A2, dCATH-A3, dCATH-A4, dCATH-A5, dCATH-A6, and dCATH-A7 in various solvent conditions.

FIG. 2 shows the results of analyzing the interaction of the control peptide dCATH and the novel dCATH analog peptides dCATH-A1, dCATH-A2, dCATH-A3, dCATH-A4, dCATH-A5, dCATH-A6, and dCATH-A7 (experimental groups) with the membrane of Acinetobacter baumannii using flow cytometry.

FIG. 3 shows the results of confirming the DNA-binding ability of the control peptide dCATH and the novel peptides dCATH-A6 and dCATH-A7 to intracellular bacterial DNA through electrophoresis (lanes 1 to 6 represent DNA alone, and DNA/peptide ratios of 1:1, 1:2, 1:3, and 1:4, respectively).

FIG. 4 shows the results of fluorescence staining used to confirm the intracellular localization of the novel peptide dCATH-A7.

FIG. 5 shows the time-dependent distribution of the novel peptide dCATH-A7 in mouse lung tissue.

FIG. 6 shows the activity of the novel peptide dCATH-A7 in mouse lung tissue.

### BEST MODE FOR CARRYING OUT THE INVENTION

In one aspect, the present disclosure relates to an antimicrobial peptide in which, i) the 3^{rd} or 4^{th} amino acid in the amino acid sequence of SEQ ID NO: 1 is substituted with alanine (Ala, A); ii) both the 3^{rd} and 4th amino acids are substituted with alanine (Ala, A); iii) both the 3^{rd} and 4^{th} amino acids are substituted with alanine (Ala, A), and one or more of the 8^{th}, 9^{th}, or 13^{th} amino acids are substituted with lysine (Lys, K); or iv) both the 3^{rd} and 4^{th} amino acids are substituted with alanine (Ala, A), the 8^{th}, 9^{th}, and 13^{th} amino acids are substituted with lysine (Lys, K), and the 19^{th} and 20th amino acids are deleted.

As used herein, the term “peptide” refers to a linear molecule in which amino acid residues are bound together via peptide bonds. The antimicrobial peptide may be prepared using chemical synthesis methods known in the art.

The parent peptide dCATH, which has the amino acid sequence of SEQ ID NO: 1 and is known in the art, is an antimicrobial peptide from isolated duck (Anas platyrhynchos), and can be synthesized by conventional peptide synthesis methods well known in the art. The synthesis method is not particularly limited. Preferably, the peptide may be synthesized using standard chemical synthesis methods for peptides (see: W. H. Freeman and Co., Proteins; Structures and Molecular Principles, 1983). Specifically, solution-phase peptide synthesis, solid-phase peptide synthesis, fragment condensation, and Fmoc or t-Boc chemistry may be used. More preferably, the peptide may be synthesized using solid-phase peptide synthesis (see: Merrifield, R. B., J. Am. Chem. Soc., 85, 2149, 1963), but with no limitations thereto.

In the present disclosure, the antimicrobial peptide may include an amino acid sequence selected from the group consisting of the amino acid sequences of SEQ ID NOS: 2 to 8.

In the present disclosure, the peptide having the amino acid sequence of SEQ ID NO: 2 was named “dCATH-A1,” in which the 3^{rd} amino acid of the parent peptide dCATH was substituted with alanine (A); the peptide having the amino acid sequence of SEQ ID NO: 3 was named “dCATH-A2,” in which the 4^{th} amino acid was substituted with alanine (A); the peptide having the amino acid sequence of SEQ ID NO: 4 was named “dCATH-A3,” in which both the 3^{rd} and 4th amino acids were substituted with alanine (A); the peptide having the amino acid sequence of SEQ ID NO: 5 was named “dCATH-A4,” in which the 3^{rd} and 4^{th} amino acids were substituted with alanine (A), and the 8^{th} amino acid was substituted with lysine (K); the peptide having the amino acid sequence of SEQ ID NO: 6 was named “dCATH-A5,” in which the 3^{rd} and 4^{th} amino acids were substituted with alanine (A), and the 8^{th} and 9^{th} amino acids were substituted with lysine (K); the peptide having the amino acid sequence of SEQ ID NO: 7 was named “dCATH-A6,” in which the 3^{rd} and 4^{th} amino acids were substituted with alanine (A), and the 8^{th}, 9^{th}, and 13^{th} amino acids were substituted with lysine (K); and the peptide having the amino acid sequence of SEQ ID NO: 8 was named “dCATH-A7,” which is an antimicrobial peptide derived from the peptide of SEQ ID NO: 7 by deleting the 19^{th} and 20^{th} amino acids.

The antimicrobial peptides may be chemically synthesized. In the case of chemical synthesis, the peptides can be obtained by using polypeptide synthesis methods well known in the art. Polypeptides may be prepared using conventional stepwise liquid-phase or solid-phase synthesis, fragment condensation, and Fmoc or t-Boc chemistry.

In addition, the antimicrobial peptides may also be obtained using recombinant DNA technology. In the case of recombinant DNA technology, a polynucleotide (nucleic acid) encoding the novel peptide of the present disclosure is inserted into an appropriate expression vector, which is then introduced into a host cell through transformation, and the host cell is cultured to express the novel peptide. The protein can then be recovered from the host cell. After expression in the selected host cell, the protein (polypeptide) may be isolated and purified using conventional biochemical separation techniques, such as treatment with protein precipitants (salting-out), centrifugation, ultrasonic disruption, ultrafiltration, dialysis, molecular sieve chromatography (gel filtration), adsorption chromatography, ion exchange chromatography, affinity chromatography, and the like. Typically, combinations of these methods are used to isolate highly pure proteins (polypeptides).

The antimicrobial peptide may have an amidated C-terminus.

The antimicrobial peptide of the present disclosure may exhibit antimicrobial activity against Gram-negative bacteria, Gram-positive bacteria, or antibiotic-resistant bacteria.

The Gram-negative bacteria are preferably those known in the art and include Gram-negative bacteria belonging to the genera Pseudomonas, Escherichia, Acinetobacter, Salmonella, Leptospira, and Rickettsia. More preferably, the Gram-negative bacteria are selected from the group consisting of Escherichia, Pseudomonas, and Acinetobacter. Most preferably, the bacteria are Escherichia coli, Pseudomonas aeruginosa, or Acinetobacter baumannii, but with no limitations thereto.

The Gram-positive bacteria are preferably those known in the art and include Gram-positive bacteria belonging to the genera Staphylococcus, Listeria, Corynebacterium, Lactobacillus, and Bacillus. More preferably, the Gram-positive bacteria are selected from the group consisting of Staphylococcus, Listeria, and Bacillus. Most preferably, the bacteria are Staphylococcus aureus, Listeria monocytogenes, or Bacillus cereus, but with no limitations thereto.

The antibiotic-resistant bacteria are preferably selected from the group consisting of Pseudomonas aeruginosa, Escherichia coli, Acinetobacter baumannii, and Staphylococcus aureus, which exhibit antibiotic resistance, but with no limitations thereto.

Examples of the antibiotics include, but are not limited to, β-lactam antibiotics, aminoglycoside antibiotics, quinolone antibiotics, peptide antibiotics, glycopeptide antibiotics, tetracycline antibiotics, rifamycin antibiotics, lincosamide antibiotics, and macrolide antibiotics.

Examples of β-lactam antibiotics include penicillin antibiotics, β-lactamase inhibitor-combined penicillin antibiotics, cephalosporin antibiotics, β-lactamase inhibitor-combined cephalosporin antibiotics, carbapenem antibiotics, β-lactamase inhibitor-combined carbapenem antibiotics, monobactam antibiotics, and penem antibiotics. Preferably, the antibiotics are penicillin antibiotics, β-lactamase inhibitor-combined penicillin antibiotics, cephalosporin antibiotics, β-lactamase inhibitor-combined cephalosporin antibiotics, and carbapenem antibiotics.

Examples of penicillin antibiotics include benzylpenicillin, penicillin O, penicillin V, penicillin G, methicillin, oxacillin, cloxacillin, dicloxacillin, carbenicillin, bacampicillin, ticarcillin, azlocillin, mezlocillin, amoxicillin, sultamicillin, talampicillin, lenampicillin, cyclacillin, pivmecillinam, aspoxicillin, ampicillin, and piperacillin. Preferably, piperacillin may be used.

Examples of β-lactamase inhibitor-combined penicillin antibiotics include ampicillin-sulbactam, ticarcillin-clavulanic acid, and piperacillin-tazobactam combinations.

Examples of cephalosporin antibiotics include cefazolin, cephalothin, cephapirin, cephalexin, cefadroxil, cephaloridine, cephtezole, cefroxadine, cefamandole, cefuroxime, cephalonium, ceforanide, cefaclor, cefprozil, cefpodoxime, loracarbef, ceftriaxone, cefotaxime, ceftizoxime, cefoperazone, cefoselis, ceftibuten, cefixime, ceftetamet, cefditoren pivoxil, cefpirome, cefoxitin, cefotetan, cefmetazole, cefbuperazone, cefminox, latamoxef, flomoxef, cefotiam, cefpiramide, cefmenoxime, cefozopran, cephatrizin, cefdinir, cefteram pivoxil, cefcapene pivoxil, ceftolozane, ceftaroline, cephradine, ceftolozane, ceftazidime, and cefepime. Preferably, cefotaxime, ceftazidime, and cefepime may be used.

Examples of β-lactamase inhibitor-combined cephalosporin antibiotics include cefoperazone-sulbactam, ceftazidime-avibactam, ceftaroline-avibactam, and ceftolozane-tazobactam combinations.

Examples of carbapenem antibiotics include imipenem, panipenem, biapenem, doripenem, ertapenem, tebipenem, tomopenem, saftrinem, lenapenem, and meropenem. Preferably, imipenem and meropenem may be used.

Examples of β-lactamase inhibitor-combined carbapenem antibiotics include imipenem-MK-7655 and biapenem-RPX7009 combinations.

Examples of monobactam antibiotics include aztreonam and carumonam.

Examples of penem antibiotics include faropenem and sulopenem.

Examples of aminoglycoside antibiotics include streptomycin, neomycin, kanamycin, paromomycin, gentamicin, tobramycin, netilmicin, spectinomycin, sisomicin, dibekacin, bekanamycin, ribostamycin, astromicin, arbekacin, plazomicin, isepamicin, and amikacin. Preferably, amikacin and gentamicin may be used.

Examples of quinolone antibiotics include nalidixic acid, oxolinic acid, piromidic acid, pipemidic acid, norfloxacin, pefloxacin, enoxacin, ofloxacin, temafloxacin, lomefloxacin, fleroxacin, grepafloxacin, sparfloxacin, trovafloxacin, clinafloxacin, gatifloxacin, moxifloxacin, sitafloxacin, garenoxacin, gemifloxacin, garenoxacin, prulifloxacin, tosufloxacin, besifloxacin, finafloxacin, delafloxacin, avarofloxacin, zabofloxacin, nemonoxacin, pazufloxacin, ciprofloxacin, and levofloxacin. Preferably, ciprofloxacin may be used.

An example of a peptide antibiotic is colistin.

Examples of glycopeptide antibiotics include vancomycin, telavancin, and teicoplanin.

Examples of tetracycline antibiotics include minocycline and tigecycline.

An example of a rifamycin antibiotic is rifampicin.

An example of a lincosamide antibiotic is clindamycin.

Examples of macrolide antibiotics include erythromycin and azithromycin.

In an embodiment of the present disclosure, the antibiotic-resistant strain may be an Acinetobacter baumannii strain resistant to one or more antibiotics selected from the group consisting of ampicillin/sulbactam combination, amikacin, aztreonam, ciprofloxacin, colistin, cefepime, gentamicin, imipenem, meropenem, minocycline, piperacillin, trimethoprim/sulfamethoxazole combination, cefotaxime, ceftazidime, ticarcillin-clavulanic acid combination, tigecycline, and piperacillin/tazobactam combination (data not shown).

In another aspect, the present disclosure relates to an antimicrobial composition including the antimicrobial peptide as an active ingredient.

As used herein, the term “antimicrobial composition” may refer to an antibiotic in the broad sense of the term, encompassing antimicrobial agents, antibiotics, preservatives, stabilizers, or sterilizing agents. It refers to a substance capable of inhibiting or suppressing the growth and biological function of pathogenic microorganisms, including, for example, Gram-negative bacteria such as Escherichia coli, Pseudomonas aeruginosa, and Acinetobacter baumannii; Gram-positive bacteria such as Staphylococcus aureus, Listeria monocytogenes, and Bacillus cereus; or antibiotic-resistant bacteria.

Escherichia coli is a Gram-negative bacterium that resides in the intestines of humans and animals and is commonly found in environments contaminated by feces. Therefore, it is often used as an indicator of contamination in items or places requiring hygiene, such as food, beverages, and restaurants.

Pseudomonas aeruginosa is a Gram-negative bacillus with relatively weak pathogenicity in humans, but it is widely distributed in natural environments. It can cause mixed or secondary infections, thereby exacerbating disease conditions. In particular, in cases where the body's resistance is compromised due to surgery or burns, it can cause sepsis (superinfection, opportunistic infection). It is commonly found in chronic otitis media, and it can invade the gastrointestinal mucosa to cause diarrhea.

Acinetobacter baumannii is a representative pathogenic Gram-negative bacterium. First identified in 1968, it has weak pathogenicity and is widely distributed in natural environments such as soil and water, as well as in hospital environments. Infections caused by Acinetobacter baumannii strains resistant to three or more classes of antibiotics (multiple drug-resistant A. baumannii, MDRAB) have surged over the past 15 years and became particularly notorious during the Iraq conflict, earning the nickname “Iraqibacter.”

Staphylococcus aureus is a Gram-positive facultative anaerobe commonly found on the skin and nasal surfaces of healthy humans and livestock. It produces heat-resistant exotoxins that cause food poisoning. It also secretes leukocidins (toxins that destroy phagocytes), hemolysins, and coagulases that help it evade host immune defenses and cause suppurative infections. Staphylococci are found on the skin or in the nasal cavity of over 50% of healthy humans and animals, and can cause a wide range of diseases from mild skin infections like acne and impetigo to life-threatening conditions such as pneumonia, meningitis, and sepsis. It is one of the five most common causes of hospital-acquired infections and frequently causes post-surgical wound infections. It is estimated that about 500, 000 patients annually in U.S. hospitals are infected with Staphylococcus aureus. Different strains of S. aureus can produce various toxins, which are classified into three types: pyrogenic toxin superantigens, exfoliative toxins, and other toxins. Strains that produce the seven types of enterotoxins responsible for food poisoning belong to the pyrogenic toxin superantigen group. The enterotoxins produced by S. aureus are the cause of gastroenteritis, leading to symptoms such as nausea, vomiting, diarrhea, and abdominal pain, which typically resolve naturally within 8-24 hours. Since the food poisoning symptoms are caused not by the bacteria itself but by the heat-stable enterotoxins, contaminated food cannot be made safe through cooking; therefore, the best precaution is to avoid consumption of food contaminated with S. aureus.

Listeria monocytogenes is the only pathogenic species among the Gram-positive rod-shaped Listeria bacteria that can infect humans. It is the causative agent of foodborne gastrointestinal infections that can be transmitted through contaminated food. The bacterium can cause serious infections such as central nervous system infections and sepsis, particularly in elderly individuals, organ transplant recipients, and patients with compromised cell-mediated immunity, such as those with AIDS.

Bacillus cereus is a Gram-positive, facultative anaerobic rod-shaped bacterium that forms heat-resistant spores. When favorable conditions are met following food production, processing, or cooking, the bacterium can proliferate rapidly, causing spoilage and deterioration. If cooked food is left at room temperature for extended periods, the spores of B. cereus may proliferate or produce toxins. Consumption of food contaminated with B. cereus may result in food poisoning symptoms such as diarrhea or vomiting due to enterotoxins produced by the bacterium.

The antimicrobial peptides of the present disclosure exhibit excellent antimicrobial activity against Escherichia coli, Pseudomonas aeruginosa, Acinetobacter baumannii, Staphylococcus aureus, Listeria monocytogenes, Bacillus cereus, and antibiotic-resistant bacteria. Therefore, the composition of the present disclosure including the antimicrobial peptide as an active ingredient can be usefully applied as an antimicrobial composition.

In particular, cytotoxicity and hemolytic activity analyses revealed that the antimicrobial peptides of the present disclosure exhibited significantly reduced cytotoxicity and hemolytic activity compared to the parent peptide (dCATH), thereby providing a safety advantage for use in the human body. While the duck-derived parent peptide dCATH has limited therapeutic application due to its cytotoxicity toward mammalian cells, the antimicrobial peptides of the present disclosure overcome such limitations.

Accordingly, the antimicrobial composition including the antimicrobial peptide with the above-described characteristics as an active ingredient can be used for various purposes and applications requiring antimicrobial activity. Specifically, it may be used not only in antibiotics, pharmaceuticals, cosmetics, food, feed, and biopesticides, but also as a material for coatings, packaging, preservatives (e.g., cosmetic, food, or pharmaceutical preservatives), and additives (e.g., pharmaceutical additives, food additives, cosmetic additives, or feed additives). For example, in medicine, it may be used as an antibiotic or contamination inhibitor; in food, as a preservative or antimicrobial agent; in agriculture, for antimicrobial, sterilizing, or disinfecting purposes; and in cosmetics or daily goods, for purposes such as dandruff suppression, athlete's foot prevention, underarm deodorization, or coating on items requiring antimicrobial properties. However, its use is not limited to these examples. The antimicrobial peptide of the present disclosure may be included in an amount of 0.00001 to 50.0% by weight based on the total weight of the antimicrobial composition. In addition, the antimicrobial composition may further comprise other known antimicrobial substances having antimicrobial or preservative activity, in addition to the antimicrobial peptide of the present disclosure.

Specifically, the antimicrobial composition of the present disclosure may be targeted toward bacteria belonging to the genera Pseudomonas, Acinetobacter, Staphylococcus, Listeria, Bacillus, and antibiotic-resistant bacteria. More specifically, the antimicrobial composition may be targeted toward Escherichia coli, Pseudomonas aeruginosa, Acinetobacter baumannii, Staphylococcus aureus, Listeria monocytogenes, Bacillus cereus, and/or antibiotic-resistant Acinetobacter baumannii, but is not limited thereto.

Hereinafter, the antimicrobial composition of the present disclosure is described in more detail by further categorizing its applications.

First, the antimicrobial composition of the present disclosure may be a pharmaceutical composition.

The antimicrobial pharmaceutical composition of the present disclosure is intended for the prevention or treatment of infectious diseases, and the infectious disease may be caused by Escherichia coli, Pseudomonas aeruginosa, Acinetobacter baumannii, Staphylococcus aureus, Listeria monocytogenes, Bacillus cereus, and/or antibiotic-resistant Acinetobacter baumannii strains.

Specifically, the infectious diseases may include, but are not limited to, one or more selected from the group consisting of food poisoning, impetigo, cellulitis, scalded skin syndrome, mastitis, bacteremia, sepsis, staphylococcal pneumonia, endocarditis, osteomyelitis, staphylococcal sepsis, toxic shock syndrome, hospital-acquired pneumonia, urinary tract infections, systemic infections (bacteremia and sepsis), skin and soft tissue infections, surgical infections, intra-abdominal infections, pulmonary infections (including those in patients with cystic fibrosis), complications associated with Helicobacter pylori (including peptic ulcers and gastric cancer), diabetic foot infections, osteomyelitis, and central nervous system infections.

As used herein, the term “prevention” refers to any action that suppresses or delays the onset of bacterial diseases caused by Gram-positive bacteria, Gram-negative bacteria, or antibiotic-resistant bacteria by administering the antimicrobial peptide of the present disclosure to a subject.

As used herein, the term “treatment” refers to any action that alleviates or: beneficially changes the symptoms of a disease by administering the antimicrobial peptide of the present disclosure to a subject suspected of having a bacterial disease caused by Gram-positive bacteria, Gram-negative bacteria, or antibiotic-resistant bacteria.

As used herein, the term “subject” refers to any animal, including humans, who has developed or is at risk of developing a bacterial disease caused by Gram-positive bacteria, Gram-negative bacteria, or antibiotic-resistant bacteria. The animal may be a mammal in need of treatment for similar symptoms, including, but not limited to, cattle, horses, sheep, pigs, goats, camels, antelopes, dogs, and cats.

The antimicrobial pharmaceutical composition of the present disclosure may be prepared by including, in addition to the active ingredient described above, one or more pharmaceutically acceptable carriers. Examples of carriers that may be used in the present disclosure include slowly metabolizable macromolecules such as liposomes, polysaccharides, polylactic acid, polyglycolic acid, polymeric amino acids, and amino acid copolymers. Examples include pharmaceutically acceptable salts of inorganic acids such as hydrochloride, hydrobromide, phosphate, and sulfate; and salts of organic acids such as acetate, propionate, malonate, and benzoate; as well as liquids such as water, saline, glycerol, and ethanol; and auxiliary substances such as hydrating agents, emulsifiers, or pH buffers. For further information on pharmaceutically acceptable carriers, reference may be made to [Remington's Pharmaceutical Sciences, Mack Publishing Company, 1991].

The antimicrobial pharmaceutical composition of the present disclosure may be formulated, using conventional methods in the pharmaceutical field, into unit dosage forms suitable for administration to the human body. Preferably, it may be formulated into dosage forms suitable for administration of peptide-based drugs. The composition may be administered via commonly used methods in the field, including oral administration or non-oral routes such as transdermal, intravenous, intramuscular, intraarterial, intraosseous, intrathecal, intraventricular, pulmonary, subcutaneous, intraperitoneal, intranasal, gastrointestinal, topical, sublingual, intravaginal, or rectal administration, but is not limited thereto.

The antimicrobial pharmaceutical composition of the present disclosure may be administered via non-oral routes during clinical administration and may be used in the form of conventional pharmaceutical formulations.

That is, the antimicrobial peptide of the present disclosure may actually be administered in various non-oral dosage forms. When formulated, it may be prepared using commonly used diluents or excipients such as fillers, extenders, binders, wetting agents, disintegrants, and surfactants. Formulations for non-oral administration include sterile aqueous solutions, non-aqueous solvents, suspensions, emulsions, lyophilized preparations, and suppositories. Examples of non-aqueous solvents and suspending agents include injectable esters such as propylene glycol, polyethylene glycol, vegetable oils such as olive oil, and ethyl oleate. Examples of bases for suppositories include witepsol, macrogol, Tween 61, cacao butter, laurin butter, and glycerogelatin.

In the pharmaceutical composition of the present disclosure, the effective dose of the antimicrobial peptide as an active ingredient is from 0.1 to 20 mg/kg, preferably from 0.5 to 10 mg/kg, and may be administered 1 to 3 times per day.

The total effective dose of the antimicrobial peptide in the pharmaceutical composition of the present disclosure may be administered as a single dose in the form of a bolus or via infusion over a relatively short period of time, or may be administered as multiple doses through a fractionated treatment protocol over an extended period. The concentration of the antimicrobial peptide and the effective dose for a patient are determined in consideration of various factors including the route of administration, frequency of treatment, age, and health condition of the patient. Therefore, those skilled in the art will be able to determine the appropriate effective dose for a specific use of the peptide as a pharmaceutical composition.

In an embodiment of the present disclosure, the antimicrobial pharmaceutical composition may be an antibiotic.

In addition, the antimicrobial composition of the present disclosure may be a food composition.

The antimicrobial food composition of the present disclosure is intended for the prevention or improvement of infectious diseases. In addition to containing the antimicrobial peptide of the present disclosure as an active ingredient, such food compositions may also include additional components such as various flavoring agents or natural carbohydrates, similar to conventional food compositions.

Examples of above-mentioned natural the carbohydrates include monosaccharides such as glucose and fructose; disaccharides such as maltose and sucrose; and polysaccharides such as dextrin and cyclodextrin, as well as sugar alcohols such as xylitol, sorbitol, and erythritol. Flavoring agents may preferably include natural flavoring agents such as thaumatin, stevia extracts (e.g., rebaudioside A, glycyrrhizin), and synthetic flavoring agents such as saccharin and aspartame.

The food composition of the present disclosure may be formulated in the same manner as the pharmaceutical composition described above and may be used as a functional food or added to various types of food. Examples of foods to which the composition may be added include beverages, meat products, chocolate, processed foods, snacks, pizza, ramen, other noodle products, chewing gum, candy, ice cream, alcoholic beverages, multivitamin preparations, and dietary supplements. In addition, the food composition may further

include, in addition to the antimicrobial peptide as an active ingredient, various nutritional supplements, vitamins, minerals (electrolytes), synthetic or natural flavoring agents, coloring agents, bulking agents (e.g., cheese, chocolate), pectic acid and its salts, alginic acid and its salts, organic acids, protective colloid thickeners, pH regulators, stabilizers, preservatives, glycerin, alcohol, and carbonating agents used in carbonated beverages. The food composition of the present disclosure may also contain fruit pulp for the manufacture of natural fruit juices, fruit juice beverages, or vegetable beverages.

Meanwhile, since the antimicrobial peptide of the present disclosure, as an active ingredient, exhibits negligible cytotoxicity, it can be safely administered even over prolonged periods for the purpose of imparting antimicrobial functionality, and thus can be effectively used as a functional food composition for the prevention or improvement of infectious diseases.

In addition, the antimicrobial composition of the present disclosure may be a cosmetic composition.

The cosmetic composition of the present disclosure may include, in addition to the antimicrobial peptide, components that commonly in cosmetic are used compositions, such as conventional auxiliaries including antioxidants, stabilizers, solubilizers, vitamins, pigments, and fragrances, as well as carriers.

In the cosmetic composition of the present disclosure, the antimicrobial peptide may be added in an amount of 0.1 to 50% by weight, preferably 1 to 30% by weight, based on the total weight of the cosmetic composition.

The cosmetic composition of the present disclosure may be prepared in any formulation commonly manufactured in the art, including, for example, solutions, suspensions, emulsions, pastes, gels, creams, lotions, powders, soaps, surfactant-containing cleansers, oils, powdered foundations, emulsified foundations, wax foundations, and sprays, but is not limited thereto. More specifically, it may be prepared in the form of softening toner (skin), nourishing toner (milk lotion), nourishing cream, massage cream, essence, eye cream, cleansing cream, cleansing foam, cleansing water, mask packs, spray, or powder.

When the formulation is a paste, cream, or gel, carriers such as animal or vegetable oils, waxes, paraffin, starch, tragacanth, cellulose derivatives, polyethylene glycol, silicone, bentonite, silica, talc, or zinc oxide may be used.

When the formulation is a powder or spray, carriers such as lactose, talc, silica, aluminum hydroxide, calcium silicate, or polyamide powder may be used. In particular, in case sprays, such the of propellants as chlorofluorohydrocarbons, propane/butane, or dimethyl ether may additionally be included.

When the formulation is a solution or emulsion, carriers such as solvents, solubilizers, or emulsifiers may be used, including water, ethanol, isopropanol, ethyl carbonate, ethyl acetate, benzyl alcohol, benzyl benzoate, propylene glycol, 1,3-butylene glycol oil, glycerol fatty acid esters, polyethylene glycol, or fatty acid esters of sorbitan.

When the formulation is a suspension, carriers such as liquid diluents including water, ethanol, or propylene glycol, suspending agents such as ethoxylated isostearyl alcohol, polyoxyethylene sorbitol esters, and polyoxyethylene sorbitan esters, and other agents such as microcrystalline cellulose, aluminum metahydroxide, bentonite, agar, or tragacanth may be used.

When the formulation is a surfactant-containing cleanser, carriers such as aliphatic alcohol sulfates, aliphatic alcohol ether sulfates, monoesters of sulfosuccinic acid, isethionates, imidazolinium derivatives, methyl taurates, sarcosinates, fatty acid amide ether sulfates, alkylamidobetaines, aliphatic alcohols, fatty acid glycerides, fatty acid diethanolamides, vegetable oils, lanolin derivatives, or ethoxylated glycerol fatty acid esters may be used.

Meanwhile, the cosmetic composition according to the present disclosure may be formulated by encapsulating the antimicrobial peptide, as an active ingredient, within a nanoliposome to enhance its stability. Encapsulating the active ingredient within the nanoliposome stabilizes it, thereby resolving formulation issues such as precipitation, discoloration, or odor changes, and enhances solubility and transdermal absorption of the ingredient, allowing for the maximum manifestation of the expected effects of the active ingredient.

In addition, the antimicrobial composition of the present disclosure may be a food additive.

When the antimicrobial peptide of the present disclosure is used as a food additive, the peptide may be added as is or in combination with other food components, and may be appropriately used according to conventional methods. The amount of the active ingredient to be mixed may be appropriately determined depending on the intended use. In general, the peptide of the present disclosure may be added in an amount of 15 parts by weight or less, preferably 10 parts by weight or less, based on the raw material. However, in the case of long-term consumption, the amount may be below this range, and since there are no safety concerns, the active ingredient may also be used in amounts exceeding the above range.

There is no particular limitation on the type of food. Examples of foods to which the substance may be added include meat products, sausages, bread, chocolate, candy, snacks, confectionery, pizza, ramen, other noodles, chewing gum, dairy products including ice cream, various soups, beverages, teas, drinks, alcoholic beverages, and multivitamin preparations, and it encompasses all foods in the conventional sense.

In addition, the antimicrobial composition of the present disclosure may be a feed additive.

Since the antimicrobial peptide of the present disclosure exhibits antimicrobial activity against Escherichia coli, Pseudomonas aeruginosa, Acinetobacter baumannii, Staphylococcus aureus, Listeria monocytogenes, Bacillus cereus, and/or antibiotic-resistant Acinetobacter baumannii strains, the feed additive comprising the peptide as an active ingredient may be used as a substitute for antibiotics in animal feed.

The feed additive may be added and mixed into compound feeds for animals such as chickens, ducks, geese, pheasants, pigs, cattle, goats, dogs, and cats, but is not limited thereto.

The feed additive including the antimicrobial peptide of the present disclosure as an active ingredient may replace conventional antibiotics, suppress the growth of harmful foodborne pathogens, improve the health status of animals, enhance weight gain and meat quality in livestock, and increase milk production and immunity.

The feed additive of the present disclosure may further include an excipient. The excipient is an ingredient added to provide appropriate hardness or shape to the formulation or, when the amount of the main ingredient is small, to give it a certain volume or weight to make it easier to handle. Without limitation, the excipient may include one or more selected from defatted rice bran, wheat bran, corn powder, cereal starch, silica powder, and diatomaceous earth, and may also include other excipients approved under the Feed Additive Standards and Specifications (Notification No. 2014-106 of the Ministry of Agriculture, Food and Rural Affairs, Korea).

In addition, the antimicrobial composition of the present disclosure may be a food preservative, cosmetic preservative, or pharmaceutical preservative.

The food preservatives, cosmetic preservatives, and pharmaceutical preservatives are additives used to prevent deterioration, spoilage, discoloration, and chemical changes in foods or pharmaceuticals. These may include sterilizing agents and antioxidants, and also include functional antibiotics that inhibit the growth of microorganisms such as bacteria, fungi, and yeasts, thereby preventing or sterilizing spoilage microorganisms in foods and pharmaceuticals. Ideally, such preservatives should be non-toxic and effective even in small amounts.

The antimicrobial peptide of the present disclosure not only exhibits excellent antimicrobial activity against Escherichia coli, Pseudomonas aeruginosa, Acinetobacter baumannii, Staphylococcus aureus, Listeria monocytogenes, Bacillus cereus, and/or antibiotic-resistant Acinetobacter baumannii strains, but also has negligible toxicity, making it useful as a preservative in foods, cosmetics, or pharmaceuticals.

The food preservative may contain the antimicrobial peptide of the present disclosure in an amount of 0.01 to 50% by weight based on the total weight of the food preservative, but is not limited thereto.

In the formulation examples of the present disclosure, the food preservative may be prepared by including, in addition to the antimicrobial peptide of the present disclosure, one or more known food preservatives. Examples of food preservatives include dehydroacetic acid, potassium sorbate, calcium sorbate, sodium benzoate, potassium benzoate, calcium benzoate, methyl parahydroxybenzoate, propyl parahydroxybenzoate, sodium propionate, and calcium propionate, but are not limited thereto.

The cosmetic preservative according to the formulation examples of the present disclosure may be prepared in the form of general emulsion or solubilized formulations. Examples of cosmetic emulsions include nourishing lotions, creams, and essences, and an example of a solubilized formulation is softening toner.

In addition, the cosmetic preservative of the present disclosure may be formulated in the form of an adjunct suitable for topical or systemic application, as commonly used in the dermatological field, by including a dermatologically acceptable medium or base in addition to the antimicrobial peptide of the present disclosure.

Suitable cosmetic formulations include, for example, solutions, gels, solid or semi-solid anhydrous products, emulsions prepared by dispersing an oil phase in a water phase, suspensions, microemulsions, microcapsules, microspheres, or vesicle dispersions in ionic (liposomal) or non-ionic form, creams, toners, lotions, powders, ointments, sprays, or concealer sticks. The composition may also be formulated as a foam or as an aerosol composition further including a compressed propellant.

The cosmetic preservative according to the formulation examples of the present disclosure may be prepared in the form of general emulsion or solubilized formulations. Examples of cosmetic emulsions include nourishing lotions, creams, and essences, and an example of a solubilized formulation is softening toner.

In addition, the antimicrobial composition of the present disclosure may be a biopesticide.

The antimicrobial composition of the present disclosure may also be a non-pharmaceutical (quasi-drug) composition.

When the composition of the present disclosure is used as a non-pharmaceutical (quasi-drug) composition, the antimicrobial peptide may be added as is, or used in combination with other non-pharmaceuticals or non-pharmaceutical ingredients, and may be appropriately applied according to conventional methods. The amount of the active ingredient to be mixed may be suitably determined depending on the intended use.

Although not limited thereto, the non-pharmaceutical (quasi-drug) composition of the present disclosure is preferably selected from the group consisting of disinfectants, shower foams, mouthwashes, wet wipes, cleansing soaps, hand washes, humidifier fillers, masks, ointments, patches, or filter fillers. In addition, the antimicrobial composition of the present disclosure may be a coating composition.

Since the composition of the present disclosure contains, as an active ingredient, a peptide exhibiting excellent antimicrobial activity against Escherichia coli, Pseudomonas aeruginosa, Acinetobacter baumannii, Staphylococcus aureus, Listeria monocytogenes, Bacillus cereus, and/or antibiotic-resistant Acinetobacter baumannii, it may be used as a coating agent for items requiring antimicrobial properties. For example, the composition of the present disclosure may be usefully applied as an antimicrobial coating material for items such as antimicrobial cutting boards, antimicrobial toothpaste containers, antimicrobial toothbrush cases, antimicrobial cosmetic containers, antimicrobial trash basins, antimicrobial cans, antimicrobial cups, antimicrobial bottles, antimicrobial rice containers, antimicrobial kimchi containers, antimicrobial dippers, antimicrobial packaging films, antimicrobial food storage containers, antimicrobial boxes, antimicrobial seasoning containers, antimicrobial water bottles, antimicrobial beverage bottles, antimicrobial nonwoven fabrics, antimicrobial textiles, antimicrobial dishcloths, antimicrobial mops, antimicrobial scrubbers, antimicrobial duvet covers, antimicrobial bed covers, antimicrobial placemats, antimicrobial toilet seat covers, antimicrobial grill plates, antimicrobial diapers, antimicrobial sanitary pads, antimicrobial masks, antimicrobial bandages, antimicrobial medical products, antimicrobial fruit packaging, antimicrobial flower wrapping, antimicrobial wallpapers, antimicrobial floor mats, and antimicrobial tiles.

In another aspect, the present disclosure relates to vivo antimicrobial method comprising an in administering the antimicrobial peptide to a subject.

The subject may be a mammal such as a human, cow, horse, sheep, pig, goat, camel, antelope, dog, or cat, but is not limited thereto.

### MODE FOR CARRYING OUT THE INVENTION

A better understanding of the present disclosure may be obtained through the following examples, which are set forth to illustrate, but are not to be construed to limit, the scope of the present disclosure.

### Example 1

### Synthesis, Isolation, and Purification of Novel Inventive Peptides

Based on Merrifield's liquid-phase peptide synthesis method (Merrifield, R. B., J. Am. Chem. Soc., 85, 2149, 1963), the amino acid sequence of the parent peptide dCATH, described in SEQ ID NO: 1, was modified by substituting the 3^{rd} and 4^{th} amino acid residues with alanine (Ala, A). Subsequently, the 8^{th}, 9^{th}, and 13th residues were sequentially substituted with lysine (Lys, K), and finally, the 19^{th} and 20^{th} residues were deleted to synthesize the peptide (see Table 1 below).

Specifically, for peptides designed in the present disclosure where the carboxy-terminal is amidated (NH2-form), Rink Amide MBHA Resin was used as the starting material. For peptides with a carboxy-terminal in the OH form, Fmoc (9-fluorenylmethoxycarbonyl) amino acid-Wang Resin was used as the starting material.

Peptide chain elongation by coupling of Fmoc-amino acids was performed using the DCC (N-hydroxybenzotriazole (HOBt)-dicyclohexylcarbodiimide) method. After coupling Fmoc-amino acids to the amino terminus of the peptide, the Fmoc group was removed using 20% piperidine/N-methylpyrrolidone (NMP) solution, followed by repeated washing with NMP and dichloromethane (DCM), and then drying under nitrogen gas. A mixed solution of trifluoroacetic acid (TFA), phenol, thioanisole, water, and triisopropylsilane in a ratio of 85:5:5:2. 5:2.5 (v/v) was added to remove protecting groups and cleave the peptide from the resin by reacting for 2-3 hours. The peptide was precipitated using diethyl ether and collected. The crude peptide thus obtained was purified using a reverse-phase (RP) HPLC column (Delta Pak, C18, 300 Å, 15, 19.0 mm×30 cm, Waters, USA) with an acetonitrile gradient containing 0.05% TFA. The synthetic peptides were hydrolyzed with 6N hydrochloric acid at 110° C., and the residues were concentrated under reduced pressure, dissolved in 0.02N hydrochloric acid, and the amino acid composition was measured using an amino acid analyzer (Hitachi 8500 A). The purity and molecular weight of the peptides were confirmed by MALDI mass spectrometry (Hill et al., Rapid Commun. Mass Spectrometry, 5:395, 1991).

As a result, as shown in Table 1 below, peptides having the amino acid sequences of SEQ ID NOS: 1 to 8 were synthesized with a purity of 95% or higher, and their molecular weights matched the expected values.

TABLE 1
Sequences, Molecular Weights, and Retention Times of
Synthetic Peptides

| | | SEQ ID | Retention | Molecular |
|---|---|---|---|---|
| Name | Amino Acid Sequence | NO: | Time | Weight |
| dCATH | KRFWQLVPLAIKIYRAWKRR | 1 | 22.519 | 2628.8 |
| dCATH- | KRAWQLVPLAIKIYRAWKRR | 2 | 21.179 | 2551.1 |
| A1 | | | | |
| dCATH- | KRFAQLVPLAIKIYRAWKRR | 3 | 21.033 | 2513.1 |
| A2 | | | | |
| dCATH- | KRAAQLVPLAIKIYRAWKRR | 4 | 19.993 | 2437.9 |
| A3 | | | | |
| dCATH- | KRAAQLVKLAIKIYRAWKRR | 5 | 21.306 | 2467.4 |
| A4 | | | | |
| dCATH- | KRAAQLVKKAIKIYRAWKRR | 6 | 16.953 | 2482.8 |
| A5 | | | | |
| dCATH- | KRAAQLVKKAIKKYRAWKRR | 7 | 13.63 | 2497.4 |
| A6 | | | | |
| dCATH- | KRAAQLVKKAIKKYRAWK | 8 | 13.173 | 2186.2 |
| A7 | | | | |

*The peptides are amidated at the C-terminus.

### Example 2

### Antimicrobial Activity of the Novel Peptides of the Present Disclosure

To compare the antimicrobial activity of the peptides prepared in <Example 1>, the minimum inhibitory concentration (MIC), which is the lowest concentration of cell division, was peptide that inhibits bacterial measured. Here, the minimal inhibitory concentration (MIC) refers to the lowest concentration of a substance that prevents visible bacterial growth.

Specifically, the bacterial strains listed in Table 2 below were obtained and cultured in media suitable for each strain until the mid-log phase. The cultures were then diluted to a concentration of 2×10⁴ cells/100 μL and prepared in microtiter plates (Nunc, USA). Subsequently, serial 2-fold dilutions of the peptides prepared in Example 1 were prepared, and 10 μL of each dilution was added to the wells, and the plates were incubated at 37° C. for 18 hours. Absorbance at 600 nm was measured using a microtiter plate reader (Merck ELISA reader, Germany) to determine the MIC values for each bacterial strain. The parent peptide dCATH was used as a control.

As a result, as shown in Table 3 below, peptides dCATH-A1 through dCATH-A5 exhibited generally superior antimicrobial activity against Gram-positive and Gram-negative bacterial strains compared to the parent peptide dCATH used as a control.

In particular, peptides dCATH-A6 and dCATH-A7 exhibited significantly superior antimicrobial activity against Acinetobacter baumannii compared to the control peptide dCATH, and notably superior activity against Staphylococcus aureus strains.

Furthermore, peptides dCATH-A2, dCATH-A3, and dCATH-A5 through dCATH-A7 showed remarkably improved antimicrobial activity against antibiotic-resistant Acinetobacter baumannii strains compared to the control peptide dCATH.

TABLE 2
Bacterial Strains and Their Sources

| | | | Accession |
|---|---|---|---|
| Classification | Strain Name | Source | No. |
| Gram-negative | Escherichia | American | ATCC |
| | coli | Type | 25922 |
| | | Culture | |
| | | Collection | |
| | Pseudomonas | American | ATCC |
| | aeruginosa | Type | 27853 |
| | | Culture | |
| | | Collection | |
| | Acinetobacter | Korean | KCTC 1017 |
| | baumannii | Collection | |
| | | for Type | |
| | | Cultures | |
| Gram-positive | Staphylococcus | American | ATCC |
| | aureus | Type | 25923 |
| | | Culture | |
| | | Collection | |
| | Listeria | Korean | KCTC 3710 |
| | monocytogenes | Collection | |
| | | for Type | |
| | | Cultures | |
| | Bacillus cereus | Korean | KCTC 2508 |
| | | Collection | |
| | | for Type | |
| | | Cultures | |
| Antibiotic- | Acinetobacter | Eulji | |
| resistant | baumannii | Hospital | |
| | Isolates 1-26 | | |

TABLE 3
Antibacterial Activity of Novel Peptides

| | | MIC(μM) | | | | | | | |
|---|---|---|---|---|---|---|---|---|---|
| Classification | | dCATH | dCATH-A1 | dCATH-A2 | dCATH-A3 | dCATH-A4 | dCATH-A5 | dCATH-A6 | dCATH-A7 |
| Gram (−) | Escherichia coli | 2 | 2 | 2 | 2 | 2 | 1 | 2 | 4 |
| | Pseudomonas aeruginosa | 2 | 4 | 2 | 2 | 2 | 1 | 2 | 8 |
| | Acinetobacter baumannii | 2 | 2 | 1 | 1 | 2 | 1 | 1 | 1 |
| Gram (+) | Pseudomonas aeruginosa | 2 | 2 | 1 | 2 | 2 | 1 | 0.5 | 0.5 |
| | Listeria monocytogenes | 2 | 2 | 2 | 2 | 2 | 1 | 2 | 4 |
| | Bacillus cereus | 1 | 2 | 1 | 2 | 2 | 1 | 2 | 1 |
| Antibiotic- | Acinetobacter | 2 | 2 | 2 | 1 | 2 | 1 | 1 | 1 |
| resistant | baumannii_isolate 1 | | | | | | | | |
| | Acinetobacter | 2 | 2 | 1 | 1 | 2 | 1 | 2 | 1 |
| | baumannii_isolate 2 | | | | | | | | |
| | Acinetobacter | 2 | 2 | 1 | 1 | 2 | 1 | 1 | 1 |
| | baumannii_isolate 3 | | | | | | | | |
| | Acinetobacter | 2 | 2 | 2 | 1 | 2 | 1 | 1 | 1 |
| | baumannii_isolate 4 | | | | | | | | |
| | Acinetobacter | 2 | 2 | 1 | 1 | 2 | 1 | 1 | 1 |
| | baumannii_isolate 5 | | | | | | | | |
| | Acinetobacter | 4 | 2 | 2 | 2 | 1 | 1 | 2 | 1 |
| | baumannii_isolate 6 | | | | | | | | |
| | Acinetobacter | 4 | 2 | 1 | 1 | 2 | 1 | 1 | 1 |
| | baumannii_isolate 7 | | | | | | | | |
| | Acinetobacter | 2 | 2 | 1 | 1 | 2 | 1 | 1 | 1 |
| | baumannii_isolate 8 | | | | | | | | |
| | Acinetobacter | 2 | 2 | 2 | 1 | 2 | 1 | 1 | 1 |
| | baumannii_isolate 9 | | | | | | | | |
| | Acinetobacter | 2 | 2 | 2 | 2 | 4 | 1 | 2 | 1 |
| | baumannii_isolate 10 | | | | | | | | |
| | Acinetobacter | 2 | 2 | 1 | 1 | 2 | 1 | 1 | 0.5 |
| | baumannii_isolate11 | | | | | | | | |
| | Acinetobacter | 2 | 2 | 1 | 1 | 2 | 1 | 1 | 1 |
| | baumannii_isolate 12 | | | | | | | | |
| | Acinetobacter | 2 | 2 | 1 | 1 | 2 | 1 | 1 | 1 |
| | baumannii_isolate 13 | | | | | | | | |
| | Acinetobacter | 2 | 2 | 1 | 2 | 2 | 1 | 2 | 1 |
| | baumannii_isolate 14 | | | | | | | | |
| | Acinetobacter | 2 | 2 | 1 | 2 | 2 | 1 | 1 | 1 |
| | baumannii_isolate15 | | | | | | | | |
| | Acinetobacter | 2 | 2 | 1 | 1 | 2 | 1 | 1 | 1 |
| | baumannii_isolate16 | | | | | | | | |
| | Acinetobacter | 2 | 1 | 1 | 1 | 2 | 1 | 1 | 1 |
| | baumannii_isolate 17 | | | | | | | | |
| | Acinetobacter | 2 | 2 | 1 | 1 | 2 | 1 | 1 | 1 |
| | baumannii_isolate 18 | | | | | | | | |
| | Acinetobacter | 2 | 1 | 1 | 1 | 2 | 1 | 1 | 1 |
| | baumannii_isolate 19 | | | | | | | | |
| | Acinetobacter | 2 | 1 | 1 | 1 | 2 | 1 | 1 | 1 |
| | baumannii_isolate20 | | | | | | | | |
| | Acinetobacter | 2 | 2 | 1 | 4 | 2 | 1 | 1 | 1 |
| | baumannii_isolate21 | | | | | | | | |
| | Acinetobacter | 2 | 2 | 1 | 1 | 2 | 2 | 1 | 1 |
| | baumannii_isolate 22 | | | | | | | | |
| | Acinetobacter | 2 | 1 | 1 | 1 | 2 | 1 | 1 | 1 |
| | baumannii_isolate 23 | | | | | | | | |
| | Acinetobacter | 4 | 2 | 1 | 1 | 2 | 2 | 1 | 1 |
| | baumannii_isolate 24 | | | | | | | | |
| | Acinetobacter | 2 | 2 | 1 | 1 | 2 | 1 | 2 | 1 |
| | baumannii_isolate 25 | | | | | | | | |
| | Acinetobacter | 2 | 2 | 2 | 1 | 2 | 1 | 2 | 1 |
| | baumannii_isolate 26 | | | | | | | | |

### Example 3

### Anti-Biofilm Activity of the Novel Peptides of the Present Disclosure

To compare the anti-biofilm activity of the peptides prepared in <Example 1>, the minimal biofilm inhibitory concentration (MBIC), defined as the lowest concentration of peptide at which bacterial biofilm cells do not increase over time, was measured. Here, the MBIC refers to the minimum concentration of an antimicrobial agent at which the average number of viable biofilm cells does not increase over time.

Specifically, among the bacterial strains listed in Table 2, those known to form biofilms well (Acinetobacter baumannii KCTC 2508, Acinetobacter baumannii isolate 10, and isolate 14) were cultured in the appropriate media to the mid-log phase and diluted to a cell concentration of 5×10⁴ cells/100 UL, followed by inoculation into microplates (SPL). Subsequently, the peptides prepared in <Example 1> were serially 10-fold diluted with 10 mM sodium phosphate buffer (pH 7.2), and 10 μL of each dilution was added to each well. The plates were then incubated at 37° C. for 24 hours. After completely removing the supernatant, the plates were fixed with 100% methanol for 15 minutes, stained with crystal violet staining solution for 1 hour, washed three times, and then the stain was solubilized with 95% ethanol. Absorbance was measured at 595 nm using a microplate reader to determine the MBIC values for each bacterial strain.

As a result, as shown in Table 4 below, peptides dCATH-A1 to dCATH-A7 exhibited relatively stronger biofilm inhibition activity compared to the control parent peptide dCATH. In particular, peptides dCATH-A3 and dCATH-A5 to dCATH-A7 demonstrated superior biofilm inhibition activity.

TABLE 4
Anti-Biofilm Activity of Novel Peptides

| | MBIC (μM) | | |
|---|---|---|---|
| | Acinetobacterx | Acinetobacter | Acinetobacter |
| | baumannii | baumannii_isolate | baumannii_isolate |
| Peptide | KCTC 2508 | 10 | 14 |
| dCATH | 4 | 4 | 4 |
| dCATH-A1 | 4 | 4 | 2 |
| dCATH-A2 | 4 | 2 | 2 |
| dCATH-A3 | 2 | 2 | 1 |
| dCATH-A4 | 4 | 4 | 4 |
| dCATH-A5 | 2 | 2 | 1 |
| dCATH-A6 | 2 | 2 | 2 |
| dCATH-A7 | 2 | 2 | 1 |

### Example 4

### Evaluation of Cytotoxicity of the Novel Peptides of the Present Disclosure

### <4-1> Hemolytic Activity Against Red Blood Cells

To evaluate the cytotoxicity of the peptides prepared in <Example 1>, their hemolytic activity against red blood cells was measured. Hemolysis refers to the rupture of red blood cells with the release of hemoglobin into the surrounding fluid. The cytotoxicity of the novel peptides of the present disclosure was assessed by measuring their hemolytic activity against erythrocytes.

Specifically, sheep red blood cells were diluted with PBS (pH 7.0) to a final concentration of 8%. The peptides dCATH, dCATH-A1, dCATH-A2, dCATH-A3, dCATH-A4, dCATH-A5, dCATH-A6, and dCATH-A7 were each added to the wells at concentrations of 3.13, 6.25, 12.5, 25.0, 50.0, and 100.0 μM/well and reacted at 37° C. for 1 hour. Thereafter, the samples were centrifuged at 1, 000×g, and the absorbance of hemoglobin in the supernatant was measured at 414 nm. As a control for complete cell lysis, 1% Triton X-100 (Sigma, USA) was used and incubated under the same conditions. The absorbance of this sample was set to represent 100% hemolysis, and hemolytic activity (%) for each peptide was calculated using the following Equation 1.

$$Red Blood Cell Hemolysis ( \% ) = ( Absorbance A - Absorbance B ) / ( Absorbance C - Absorbance B ) \times 100 \qquad [ Equation 1 ]$$

In the above equation, Absorbance A is the absorbance at 414 nm of the reaction solution treated with each peptide, Absorbance B is the absorbance at 414 nm of the PBS-treated reaction solution, and Absorbance C is the absorbance at 414 nm of the reaction solution treated with 1% Triton X-100.

As shown in Table 5 below, when the parent peptide dCATH and novel peptides dCATH-A1 and dCATH-A2 were tested at a concentration of 100 μM, 100% hemolysis of sheep red blood cells was observed. Peptides dCATH-A3, dCATH-A4, and dCATH-A5 induced more than 50% hemolysis, whereas peptides dCATH-A6 and dCATH-A7 showed less than 50% hemolytic activity at the same concentration (100 μM). Additionally, no hemolytic activity was observed at concentrations corresponding to their antimicrobial activity.

These results demonstrate that the novel peptides of the present disclosure exhibit reduced cytotoxicity compared to the parent peptide.

TABLE 5
Hemolytic Activity (%) of Novel Peptides

| | Hemolytic Activity (%) | | | | | |
|---|---|---|---|---|---|---|
| | 3.13 | 6.25 | 12.5 | 25 | 50 | 100 |
| Peptide | μM | μM | μM | μM | μM | μM |
| dCATH | 8.71 | 19.68 | 41.39 | 73.92 | 99.93 | 100 |
| dCATH-A1 | 0.43 | 1.71 | 4.92 | 13.02 | 53.63 | 100 |
| dCATH-A2 | 0 | 0.32 | 1.52 | 8.16 | 39.65 | 100 |
| dCATH-A3 | 0.35 | 0.9 | 3.08 | 8.43 | 23.91 | 68.97 |
| dCATH-A4 | 6.81 | 12.32 | 22.08 | 41.5 | 74.85 | 98.04 |
| dCATH-A5 | 1.44 | 1.28 | 3.31 | 8.64 | 24.9 | 69.73 |
| dCATH-A6 | 0 | 0.07 | 0.84 | 3.31 | 13.28 | 49.43 |
| dCATH-A7 | 0 | 0 | 0.46 | 2.48 | 11.76 | 47.81 |

### <4-2> MTT Assay

To evaluate the cytotoxicity of the peptides prepared in <Example 1>, toxicity was measured using a human keratinocyte cell line (HaCaT cell line, Dr. N. E. Fusenig, Heidelberg, Germany).

Specifically, HaCaT cells cultured in DMEM medium containing 10% fetal bovine serum (FBS) were seeded at a density of 2×10⁵ cells/well into a microtiter plate and incubated for 24 hours. Then, peptides dCATH, dCATH-A1, dCATH-A2, dCATH-A3, dCATH-A4, dCATH-A5, dCATH-A6, and dCATH-A7 were added to each well at concentrations of 3.13, 6.25, 12.5, 25.0, 50.0, and 100.0 μM/well, followed by incubation for 24 hours in a 5% CO₂ incubator. After 24 hours, 100 μL of a reaction solution containing 0.5 mg/mL MTT (Thiazolyl Blue Tetrazolium Bromide) dissolved in phosphate-buffered saline (PBS) was added to each well and reacted for 4 hours. After removing the supernatant, 200 μL of DMSO (dimethyl sulfoxide) was added to dissolve the formed MTT crystals, and absorbance at 570 nm was measured to determine cell viability.

As shown in Table 6 below, when the control parent peptide dCATH and novel peptides dCATH-A1 to dCATH-A5 were applied at a concentration of 100 μM, the HaCaT cells showed 0% viability, nearly indicating very high In contrast, peptides cytotoxicity of the peptides. dCATH-A6 and dCATH-A7 showed cell viabilities of 91.96% and 98.40%, respectively, at the same concentration, indicating that their cytotoxicity was very low or nearly negligible.

TABLE 6
Cell viability (%) of Human Keratinocytes
Treated with Novel Peptides

| | Cell Viability | | | | | |
|---|---|---|---|---|---|---|
| | 3.13 | 6.25 | 12.5 | 25 | 50 | 100 |
| Peptide | μM | μM | μM | μM | μM | μM |
| dCATH | 100 | 100 | 75.3 | 11.61 | 2.51 | 2.45 |
| dCATH-A1 | 100 | 100 | 97.94 | 29.98 | 0.81 | 1.79 |
| dCATH-A2 | 100 | 100 | 88.97 | 30.66 | 2.99 | 2.16 |
| dCATH-A3 | 91.96 | 91.91 | 97.89 | 56.56 | 5.47 | 1.59 |
| dCATH-A4 | 90.92 | 90.21 | 58.04 | 5.59 | 2.49 | 1.36 |
| dCATH-A5 | 100.46 | 92.29 | 95.06 | 57.98 | 8.27 | 3.71 |
| dCATH-A6 | 98.86 | 100 | 94.73 | 98.05 | 97.85 | 91.96 |
| dCATH-A7 | 98.03 | 99.22 | 100 | 100 | 99.95 | 98.4 |

### Example 5

### Stability of the Novel Peptides of the Present Disclosure Under Physiological Salt Conditions

To determine whether the peptides prepared in <Example 1> retain antimicrobial activity under salt concentrations present in vivo, the minimal inhibitory concentration (MIC) of the peptides was measured under various salt concentrations.

Specifically, among the bacterial strains listed in Table 2, Acinetobacter baumannii KCTC 2508 was cultured in suitable medium to the mid-log phase, then diluted to a concentration of 2×10⁴ cells/100 μL and dispensed into a microtiter plate (Nunc, USA). The peptides prepared in <Example 1> were serially diluted two-fold under various salt concentration conditions and added to the wells, followed by incubation at 37° C. for 18 hours. Absorbance at 600 nm was measured using a microtiter plate reader (Merck ELISA reader, Germany) to determine the MIC values for each strain. The parent peptide dCATH was used as a control.

As shown in Table 7 below, peptides dCATH-A1 to dCATH-A5 exhibited antimicrobial activity similar to that of the parent peptide dCATH, whereas peptides dCATH-A6 and dCATH-A7 exhibited lower MIC values than the control, indicating superior antimicrobial activity.

These results confirm that the novel peptides dCATH-A6 and dCATH-A7 exhibit strong antimicrobial activity even under various salt conditions, and in particular, show improved antimicrobial activity compared to the parent peptide dCATH used as the control.

TABLE 7
Antibacterial Activity of Inventive Novel Peptides Under Various Salt Conditions

| | | | MIC (μM) | | | | | | | |
|---|---|---|---|---|---|---|---|---|---|---|
| Salt | | | dCATH | dCATH-A1 | dCATH-A2 | dCATH-A3 | dCATH-A4 | dCATH-A5 | dCATH-A6 | dCATH-A7 |
| SP buffer | 10 | mM | 2 | 2 | 2 | 2 | 2 | 1 | 1 | 1 |
| NaCl | 10 | mM | 4 | 4 | 2 | 2 | 4 | 4 | 2 | 4 |
| | 50 | mM | 2 | 2 | 2 | 2 | 2 | 1 | 1 | 1 |
| | 100 | mM | 2 | 1 | 1 | 1 | 2 | 1 | 1 | 1 |
| MgCl₂ | 150 | mM | 2 | 4 | 2 | 2 | 4 | 2 | 1 | 1 |
| | 0.5 | mM | 4 | 2 | 2 | 2 | 4 | 2 | 1 | 2 |
| | 1 | mM | 4 | 2 | 2 | 2 | 2 | 2 | 2 | 1 |
| FeCl₃ | 2 | mM | 2 | 2 | 2 | 2 | 4 | 2 | 1 | 1 |
| | 3 | mM | 4 | 2 | 2 | 2 | 4 | 2 | 1 | 1 |
| | 4 | mM | 4 | 2 | 2 | 2 | 4 | 1 | 1 | 1 |

### Example 6

Circular Dichroism Spectroscopy Measurement To determine whether the peptides prepared in <Example 1> form a secondary x-helical structure, circular dichroism (CD) spectroscopy was performed.

Specifically, peptides dCATH, dCATH-A1, dCATH-A2, dCATH-A3, dCATH-A4, dCATH-A5, dCATH-A6, and dCATH-A7 were added at a concentration of 40 μM to 10 mM sodium phosphate buffer (pH 7.4), 50% 2, 2, 2-trifluoroethanol (TFE), or 30 mM sodium dodecyl sulfate (SDS) solutions. The peptide solutions were placed in a cuvette with a 0.1 cm path-length, and circular dichroism spectra were recorded at 25° C. using a Jasco 810 spectrophotometer. The x-helical structure for the circular dichroism spectra was calculated using Equation 2 below.

$$[ θ ] = \frac{{θ}_{obs}}{10 \cdot l \cdot c} \qquad [ Equation 2 ]$$

wherein, θ_{obs} represents the observed signal in millidegrees, 1 denotes the optical path-length of the cuvette in centimeters, and c is the peptide concentration in mol/L.

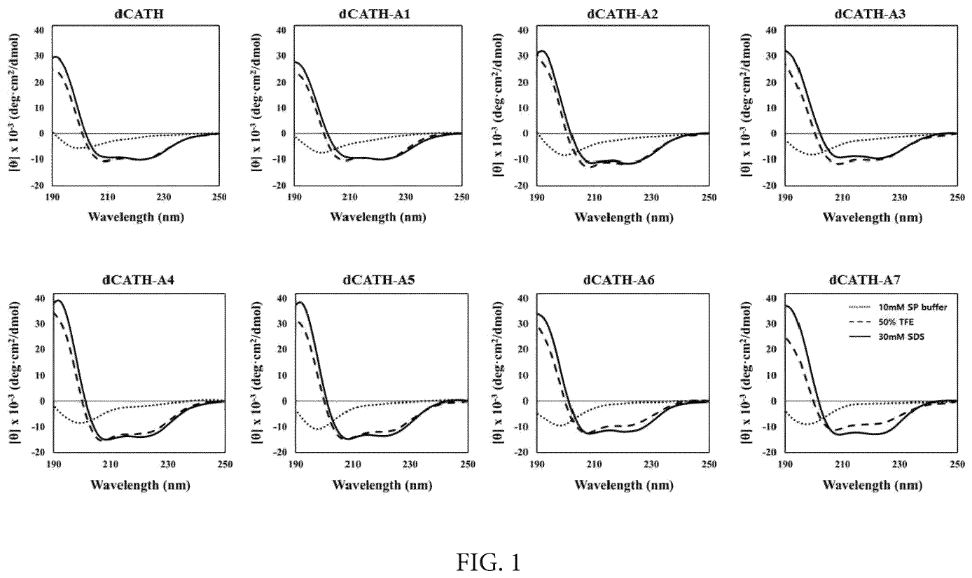

As shown in FIG. 1, no structures were observed when the peptides were added to 10 mM sodium phosphate buffer. In contrast, when the peptides were added to 50% TFE solution or 30 mM SDS solution, all peptides formed a-helical secondary structures to varying degrees.

### Example 7

### Flow Cytometry Analysis

To determine whether the peptides prepared in <Example 1> act on bacterial membranes, the parent peptide and the novel peptides dCATH-A1, dCATH-A2, dCATH-A3, dCATH-A4, dCATH-A5, dCATH-A6, and dCATH-A7 were analyzed using flow cytometry.

Specifically, Acinetobacter baumannii was treated with each peptide at twice the minimum inhibitory concentration (MIC), followed by incubation at 37° C. for 10 minutes. The supernatant was then removed by centrifugation at 10,000 rpm, and the bacterial cells were stained with propidium iodide (PI) at a concentration of 10 μg/mL at 4° C. for 30 minutes. Unbound PI was removed by centrifugation, and 1 mL of phosphate-buffered saline (PBS) was added to disperse any cell clumps. The effect of the peptides on the bacterial membrane was then assessed using a Beckman flow cytometer.

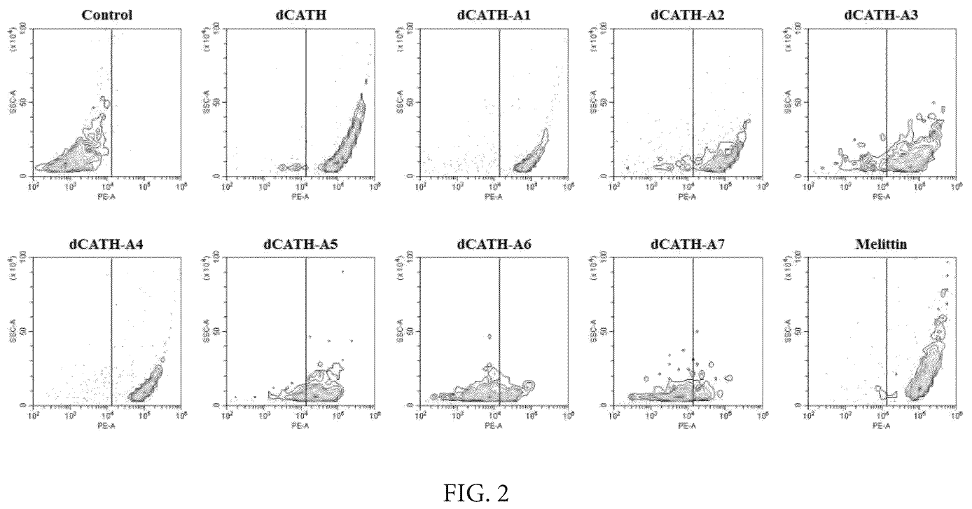

As shown in FIG. 2, the parent peptide dCATH and the novel peptides dCATH-A1 to dCATH-A5 induced membrane damage in Acinetobacter baumannii, as indicated by a rightward shift in the fluorescence signal. In contrast, dCATH-A6 and dCATH-A7 did not cause any shift in the fluorescence signal, indicating no damage to the bacterial membrane.

These results suggest that the novel peptides dCATH-A6 and dCATH-A7 exert their antimicrobial effects via a mechanism different from that of the parent peptide.

### Example 8

### Analysis of Binding Between Inventive Novel Peptides and Bacterial DNA

As described in <Example 7>, it was confirmed that the novel peptides dCATH-A6 and dCATH-A7 of the present disclosure do not act on the bacterial membrane. Accordingly, to further elucidate the mechanism by which these novel peptides exert antimicrobial activity, it was examined whether the novel peptides dCATH-A6 and dCATH-A7 bind to intracellular bacterial DNA using electrophoresis.

Specifically, 260 ng of plasmid DNA (pRSETB) was reacted with each peptide at various ratios (lanes 1 through 6 represent DNA only, 1:1, 1:2, 1:3, and 1:4 DNA/peptide ratios, respectively) at 37° C. for 10 minutes. The samples were then subjected to electrophoresis on a 1% agarose gel, stained with ethidium bromide (EtBr), and visualized under UV light. Buforin 2, an antimicrobial peptide known to bind bacterial DNA and exhibit antimicrobial activity, was used as a positive control.

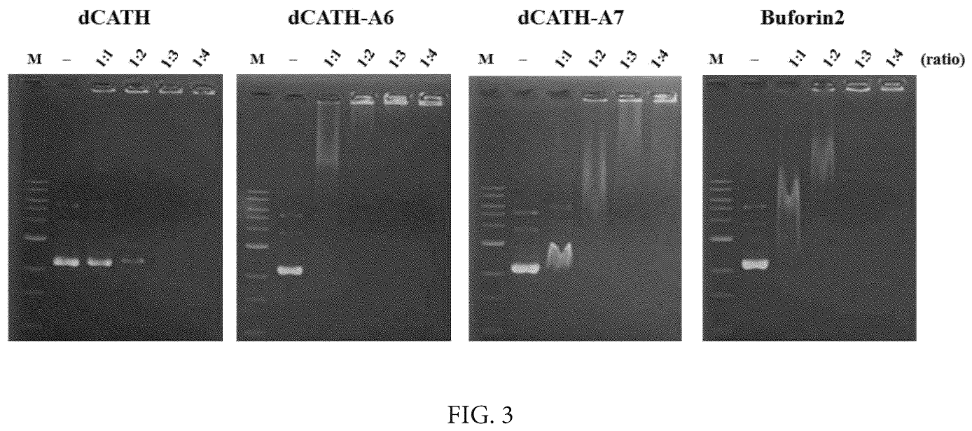

As shown in FIG. 3, while the parent peptide dCATH showed no detectable binding to DNA, it was confirmed that the novel peptides dCATH-A6 and dCATH-A7 of the present disclosure did bind to DNA.

From the above results, it was confirmed that the novel peptides dCATH-A6 and dCATH-A7, due to substitution or deletion of certain amino acid residues, exert antimicrobial activity by binding to intracellular bacterial DNA, unlike the parent peptide dCATH, which exhibits antimicrobial activity via interaction with the bacterial membrane.

### Example 9

### Intracellular Localization of Inventive Novel Peptide

Among the peptides prepared in <Example 1>, the peptide dCATH-A7, which exhibited no cytotoxicity and excellent activity, was selected for intracellular localization analysis. The cells used in the experiment were MRC-5 human lung fibroblast cells, cultured in RPMI medium supplemented with 10% fetal bovine serum (FBS).

MRC-5 cells were seeded into a 24-well plate at a density of 5×10⁵ cells/well and incubated for 24 hours. FITC-labeled dCATH-A7 was then added at a concentration of 2 μM/well and incubated for 1 hour in a 5% CO₂ incubator. After the reaction, the cells were washed with 200 μL of phosphate-buffered saline (PBS), stained with Hoechst 33342 for 10 minutes, fixed with 1% glutaraldehyde solution for 10 minutes, and washed again with 200 μL of PBS. Intracellular localization was observed using the EVOS FL Auto 2 cell imaging system.

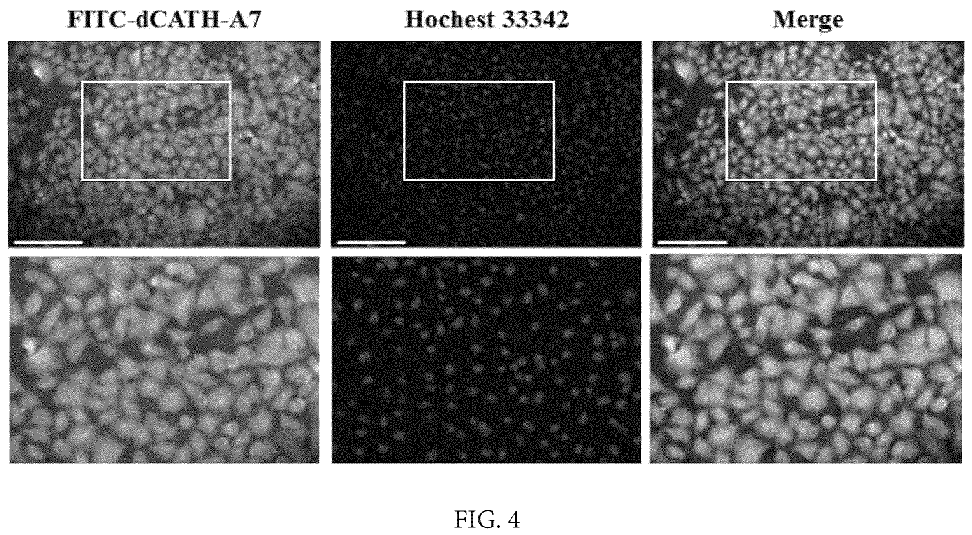

As shown in FIG. 4, it was confirmed that the FITC-dCATH-A7 peptide was internalized and localized within the MRC-5 cells.

From these results, the mode of action of the dCATH-A7 peptide was elucidated.

### Example 10

### Post-Nasal Inhalation Localization of Inventive Novel Peptide

To verify whether the peptide dCATH-A7, prepared in <Example 1>, localizes to the lungs after intranasal inhalation, the peptide was administered nasally in mice.

After a one-week acclimation period, BALB/c mice (6 weeks old) were administered 20 μL of FITC-labeled dCATH-A7 peptide intranasally at a dose of 5 mg/kg. Lung tissues were sampled at 10 minutes, 20 minutes, 30 minutes, 1 hour, 2 hours, and 24 hours post-administration, and the fluorescence intensity of FITC was measured using the FOBI in vivo imaging system.

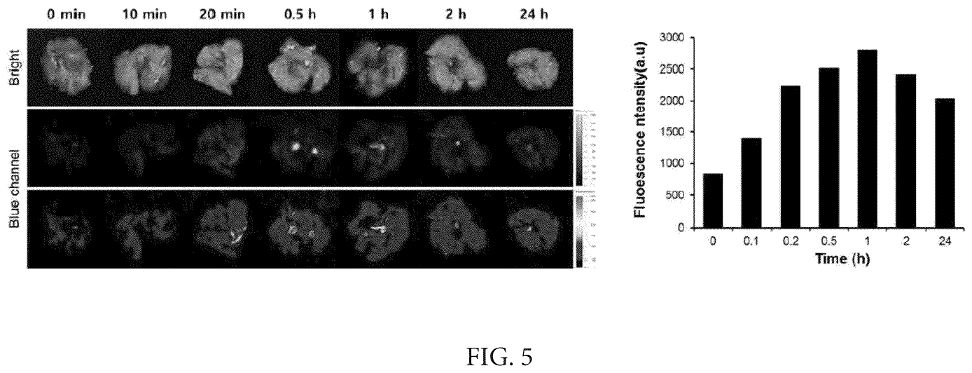

As shown in FIG. 5, FITC fluorescence was low at 0 minutes (approximately 700 a.u.) but increased to approximately 1500 a.u. at 10 minutes and reached about 3000 a.u. at 1 hour, confirming the presence of the peptide within the lung tissue. Fluorescence signals were still detectable up to 24 hours post-administration.

These results indicate that the dCATH-A7 peptide remains localized and active within the lung tissue for up to 24 hours following nasal inhalation.

### Example 11

### In Vivo Activity of the Novel Peptide of the Present Disclosure

Among the peptides prepared in <Example 1>, dCATH-A7 was selected to evaluate in vivo antimicrobial activity.

Among the antibiotic-resistant strains, Acinetobacter baumannii isolate 10 was selected for the experiment. The bacteria were washed three times with PBS buffer prior to use. After a one-week acclimation period, BALB/c mice (6 weeks old) were intranasally infected with 20 μL of a 1×10⁹ CFU/mL bacterial suspension. Following infection, the peptide was prepared at a concentration of 5 mg/mL and administered intranasally. On day 3, lung tissue samples were collected and subjected to hematoxylin and eosin (H&E) staining to assess tissue damage.

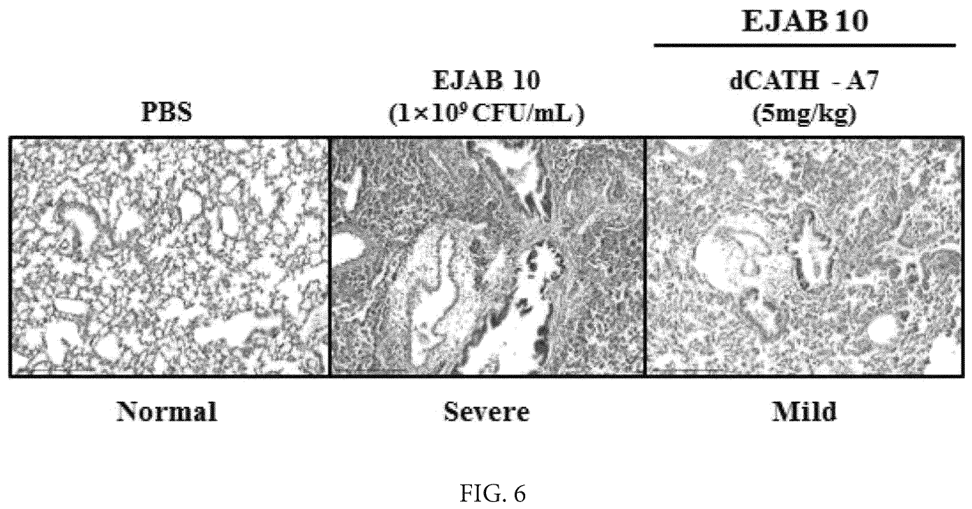

As shown in FIG. 6, lung tissues from mice infected with the antibiotic-resistant strain exhibited alveolar destruction, infiltration of immune cells, and evidence of fibrosis. In contrast, lung tissues from mice treated with the novel peptide dCATH-A7 along with the antibiotic-resistant bacteria showed minimal fibrosis and overall reduced tissue damage.

These results demonstrate that the dCATH-A7 peptide exhibits antimicrobial activity in vivo.

The following provides exemplary Preparation Examples for the composition of the present disclosure.

### Preparation Example 1

### Preparation of Pharmaceutical Formulations

### <1-1> Preparation of Powder Formulation

Novel peptide of the present disclosure: 20 mg
Lactose: 20 mg

The above ingredients were mixed and loaded into airtight pouches to prepare a powder formulation.

### <1-2> Preparation of Tablet Formulation

Novel peptide of the present disclosure: 10 mg
Corn starch: 100 mg
Lactose: 100 mg
Magnesium stearate: 2 mg

The above ingredients were mixed and compressed into tablets according to conventional tablet manufacturing methods.

### <1-3> Preparation of Capsule Formulation

Novel peptide of the present disclosure: 10 mg
Crystalline cellulose: 3 mg
Lactose: 14.8 mg
Magnesium stearate: 0.2 mg

The above ingredients were mixed and loaded into gelatin capsules according to conventional capsule manufacturing methods.

### <1-4> Preparation of Liquid Formulation

Novel peptide of the present disclosure: 20 mg
Isomerized sugar: 10 g
Mannitol: 5 g
Purified water: q.s.

According to conventional liquid formulation manufacturing methods, each ingredient was dissolved in purified water, an appropriate amount of lemon flavoring was added, and the solution was adjusted to a total volume of 100 mL with purified water. The final solution was loaded into brown bottles and sterilized to produce the liquid formulation.

### <1-5> Preparation of Injectable Formulation

Novel peptide of the present disclosure: 10 μg/mL
Dilute hydrochloric acid BP: to adjust pH to 7.6
Injectable sodium chloride: up to 1 mL

The novel peptide of the present disclosure was dissolved in an appropriate volume of Injectable sodium chloride BP. The pH of the resulting solution was adjusted to 7.6 using dilute hydrochloric acid BP, and the volume was adjusted with injectable sodium chloride BP, followed by thorough mixing. The solution was filled into 5 mL Type I clear glass ampoules, sealed under a headspace of air by melting the glass, and sterilized in an autoclave at 120° C. for at least 15 minutes to obtain an injectable formulation.

### Preparation Example 2

### Preparation of Cosmetics

### <2-1> Softening Toner (Skin Lotion)

As shown in the following table, a softening lotion was prepared according to a conventional method.

TABLE 8
Formulation Example of Softening Toner

| Ingredient | Content (wt %) |
|---|---|
| Inventive novel peptide | 0.1~30 |
| 1,3-Butylene glycol | 3 |
| Glycerin | 5 |
| Polyoxyethylene(60) Hydrogenated Castor Oil | 0.2 |
| Ethanol | 8 |
| Citric acid | 0.02 |
| Sodium Citrate | 0.06 |
| Preservative | Trace amount |
| Flavoring | Trace amount |
| Purified Water | To 100 |

### <2-2> Nutrient Toner (Lotion)

As shown in the following table, a nutrient toner was prepared according to a conventional method.

TABLE 9
Formulation Example of Nutrient Toner

| Ingredient | Content (wt %) |
|---|---|
| Inventive novel peptide | 0.1~30 |
| Squalane | 10 |
| Polyoxyethylene sorbitan monooleate | 2 |
| Guaiacum oil | 0.1~30 |
| 1,3-Butylene glycol | 8 |
| Glycerin | 5 |
| Polyoxyethylene(60) Hydrogenated Castor Oil | 0.2 |
| Ethanol | 8 |
| Citric acid | 0.02 |
| Sodium Citrate | 0.06 |
| Preservative | Trace amount |
| Flavoring | Trace amount |
| Purified Water | To 100 |

### <2-3> Essence

As shown in the following table, a facial essence was prepared according to a conventional method.

TABLE 10
Formulation Example of Essence

| Ingredient | Content (wt %) |
|---|---|
| Inventive novel peptide | 0.1~30 |
| Sitosterol | 1.7 |
| Polyglycerol 2-oleate | 1.5 |
| Ceramide | 0.7 |
| Ceteareth-4 | 1.2 |
| Cholesterol | 1.5 |
| Dicetyl phosphate | 0.4 |
| Conc. Glycerin | 5 |
| Carboxyvinyl polymer | 0.2 |
| Xanthan gum | 0.2 |
| Preservative | Trace amount |
| Flavoring | Trace amount |
| Purified Water | To 100 |

### <2-4> Face Wash (Cleansing Foam)

As shown in the following table, a face wash was prepared according to a conventional method.

TABLE 11
Formulation Example of Face Wash

| Ingredient | Content (wt %) |
|---|---|
| Inventive novel peptide | 0.1~30 |
| Sodium N-acylglutamate | 20 |
| Glycerin | 10 |
| PEG-400 | 15 |
| Propylene glycol | 10 |
| POE(15) Oleyl alcohol ether | 3 |
| Laurin derivative | 2 |
| Methyl paraben | 0.2 |
| EDTA-4Na | 0.03 |
| Flavoring | 0.2 |
| Purified Water | To 100 |

### <2-5> Nutrient Cream

As shown in the following table, a nutrient cream was prepared according to a conventional method.

TABLE 12
Formulation Example of Nutrient Cream

| Ingredient | Content (wt %) |
|---|---|
| Inventive novel peptide | 0.1~30 |
| Vaseline | 7 |
| Liquid paraffin | 10 |
| Beeswax | 2 |
| Polysorbate 60 | 2.5 |
| Sorbitan sesquioleate | 1.5 |
| Squalane | 3 |
| Propylene glycol | 6 |
| Glycerin | 4 |
| Triethanol amine | 0.5 |
| Xanthan gum | 0.5 |
| Tocophenyl acetate | 0.1 |
| Flavoring, Preservative | Trace amount |
| Purified Water | To 100 |

### <2-6> Massage Cream

As shown in the following table, a massage cream was prepared according to a conventional method.

TABLE 13
Formulation Example of Massage Cream

| Ingredient | Content (wt %) |
|---|---|
| Inventive novel peptide | 0.1~30 |
| Propylene glycol | 6 |
| Glycerin | 4 |
| Triethanol amine | 0.5 |
| Beeswax | 2 |
| Tocophenyl acetate | 0.1 |
| Polysorbate60 | 3 |
| Sorbitan sesquioleate | 2.5 |
| Cetearyl alcohol | 2 |
| Liquid paraffin | 30 |
| Xanthan gum | 0.5 |
| Flavoring, Preservative | Trace amount |
| Purified Water | To 100 |

### <2-7> Pack

As shown in the following table, a pack was prepared according to a conventional method.

TABLE 14
Formulation Example of Pack

| Ingredient | Content (wt %) |
|---|---|
| Inventive novel peptide | 0.1~30 |
| Propylene glycol | 2 |
| Glycerin | 4 |
| Polyvinyl alcohol | 10 |
| Ethanol | 7 |
| PEG-40 hydrogenated castor oil | 0.8 |
| Triethanol amine | 0.3 |
| Flavoring, Preservative | Trace amount |
| Purified Water | To 100 |

While the present invention has been particularly shown and described with reference to exemplary embodiments thereof, it will be understood by those of ordinary skill in the art that various changes in form and details may be made therein without departing from the spirit and scope of the present invention as defined by the following claims.

1. An antimicrobial peptide in which:
i) a 3rd or a 4th amino acid in the amino acid sequence of SEQ ID NO: 1 is substituted with alanine (A);
ii) the 3rd and 4th amino acids are substituted with alanine (A);
iii) the 3rd and 4th amino acids are substituted with alanine (A), and at least one of the 8th, 9th, or 13th amino acids is substituted with lysine (K); or
iv) the 3rd and 4th amino acids are substituted with alanine (A), the 8th, 9th, and 13th amino acids are substituted with lysine (K), and the 19th and 20th amino acids are deleted.

2. The antimicrobial peptide of claim 1, wherein the antimicrobial peptide is selected from the group consisting of peptides having the amino acid sequences of SEQ ID NOS: 2 to 8.

3. The antimicrobial peptide of claim 1, wherein the antimicrobial peptide exhibits antimicrobial activity against Gram-negative bacteria, Gram-positive bacteria, or antibiotic-resistant bacteria.

4. The antimicrobial peptide of claim 3, wherein the gram-negative bacteria are at least one selected from Escherichia coli, Pseudomonas aeruginosa, and Acinetobacter baumannii.

5. The antimicrobial peptide of claim 3, wherein the Gram-positive bacteria are at least one selected from Staphylococcus aureus, Listeria monocytogenes, and Bacillus cereus.

6. The antimicrobial peptide of claim 1, wherein the antibiotic-resistant bacteria is Acinetobacter baumannii having antibiotic resistance.

7. The antimicrobial peptide of claim 1, wherein the antimicrobial peptide has a C-terminus amidated.

8. An antibiotic comprising the antimicrobial peptide of claim 1 as an active ingredient.

9. A cosmetic composition for antimicrobial use, comprising the antimicrobial peptide of claim 1 as an active ingredient.

10. A food additive for antimicrobial use, comprising the antimicrobial peptide of claim 1 as an active ingredient.

11. A feed additive for antimicrobial use, comprising the antimicrobial peptide of claim 1 as an active ingredient.

12. A preservative for cosmetics or pharmaceuticals, comprising the antimicrobial peptide of claim 1 as an active ingredient.

13. A biopesticide for antimicrobial use, comprising the antimicrobial peptide of claim 1 as an active ingredient.

14. A quasi-drug composition for antimicrobial use, comprising the antimicrobial peptide of claim 1 as an active ingredient.

15. A method for antimicrobial treatment in a subject including administering a pharmaceutically effective amount of the antimicrobial peptide of claim 1 to the subject.


SEQUENCE LISTING (8 sequences)

SEQ ID NO: 1 - protein, 20 aa, synthetic construct
  REGION 1..20: note = dCATH amino acid sequence
KRFWQLVPLAIKIYRAWKRR

SEQ ID NO: 2 - protein, 20 aa, synthetic construct
  REGION 1..20: note = dCATH-A1 amino acid sequence
KRAWQLVPLAIKIYRAWKRR

SEQ ID NO: 3 - protein, 20 aa, synthetic construct
  REGION 1..20: note = dCATH-A2 amino acid sequence
KRFAQLVPLAIKIYRAWKRR

SEQ ID NO: 4 - protein, 20 aa, synthetic construct
  REGION 1..20: note = dCATH-A3 amino acid sequence
KRAAQLVPLAIKIYRAWKRR

SEQ ID NO: 5 - protein, 20 aa, synthetic construct
  REGION 1..20: note = dCATH-A4 amino acid sequence
KRAAQLVKLAIKIYRAWKRR

SEQ ID NO: 6 - protein, 20 aa, synthetic construct
  REGION 1..20: note = dCATH-A5 amino acid sequence
KRAAQLVKKAIKIYRAWKRR

SEQ ID NO: 7 - protein, 20 aa, synthetic construct
  REGION 1..20: note = dCATH-A6 amino acid sequence
KRAAQLVKKAIKKYRAWKRR

SEQ ID NO: 8 - protein, 18 aa, synthetic construct
  REGION 1..18: note = dCATH-A7 amino acid sequence
KRAAQLVKKAIKKYRAWK